(19) United States
(12) Patent Application Publication
(10) Pub. No.: US 2007/0193582 A1
(43) Pub. Date: Aug. 23, 2007

(54) Touchless control system for breathing apparatus

(75) Inventors: Philip Rodney Kwok, Chatswood (AU); Philip John Gunning, North Rocks (AU); Paul Anthony Green, Lindfield (AU); Robert Edward Henry, Roseville (AU); Dennis Peter Turner, Marayong (AU)

Correspondence Address: NIXON & VANDERHYE, PC, 901 NORTH GLEBE ROAD, 11TH FLOOR, ARLINGTON, VA, 22203, US

(73) Assignee: ResMed Limited, Bella Vista (AU)

(21) Appl. No.: 11/707,137

(22) Filed: Feb. 16, 2007

Related U.S. Application Data
(60) Provisional application No. 60/774,194, filed on Feb. 17, 2006.

(51) Int. Cl. A61M 16/00

(52) U.S. Cl. 128/204.18; 128/204.21

(57) ABSTRACT

An air delivery system includes a controllable flow generator operable to generate a supply of pressurized breathable gas to be provided to a patient for treatment. A touchless control system is associated with at least one control feature adapted to control at least one operating parameter of the flow generator. The touchless control system includes one or more sensors to detect patient hand movement and a controller to selectively activate the at least one control feature based on the patient hand movement detected by the one or more sensors.

### CROSS-REFERENCES TO RELATED APPLICATIONS

This application claims the benefit of U.S. Provisional Application No. 60/774,194, filed Feb. 17, 2006, which is incorporated herein by reference in its entirety.

### FIELD OF THE INVENTION

The present invention relates to a breathing apparatus that delivers breathable gas to a patient.

### BACKGROUND OF THE INVENTION

Breathing apparatus to deliver breathable gas to a patient typically includes a flow generator, an air delivery conduit, and a patient interface. In use, the air delivery conduit delivers pressurized gas from the flow generator to the patient interface in communication with the patient's upper airways for treatment, e.g., of Sleep Disordered Breathing (SDB) with Continuous Positive Airway Pressure (CPAP) or Non-Invasive Positive Pressure Ventilation (NIPPV) devices.

CPAP patients occasionally find need to get up at night and visit the bathroom or kitchen, or the need to otherwise arise. Typically, such a patient will turn off the CPAP apparatus via a power button provided to the flow generator. However, CPAP patients may have poor dexterity, e.g., due to age, weight, and/or arthritis, and pressing a button to turn the CPAP apparatus off requires a relatively high degree of coordination, especially at night when light may be scarce and the patient may be half-asleep.

A known CPAP apparatus is configured to turn on and off responsive to patient breathing, ResMed features commercially known as “Smartstart™” and “Smartstop™”. Another known CPAP apparatus is configured to turn off when the mask seal is broken. Yet another known CPAP apparatus includes a sensor, e.g., microphone, accelerometer, infrared sensor, contact sensor, to turn on an illumination device adjacent the display of the apparatus (see U.S. Publication No. 2005/0235993).

The present invention provides improvements and alternatives to known breathing apparatus to enhance and/or facilitate the treatment session.

### SUMMARY OF THE INVENTION

One aspect of the invention relates to a touchless control system to control one or more operating parameters of a breathing apparatus.

Another aspect of the invention relates to a touchless control system that detects patient input, e.g., hand movement, and selectively adjusts operation of a breathing apparatus based on the detected patient input.

Another aspect of the invention relates to an air delivery system including a controllable flow generator operable to generate a supply of pressurized breathable gas to be provided to a patient for treatment. A touchless control system is associated with at least one control feature adapted to control at least one operating parameter of the flow generator. The touchless control system includes one or more sensors to detect patient hand movement and a controller to selectively activate the at least one control feature based on the patient hand movement detected by the one or more sensors.

Another aspect of the invention relates to an air delivery system including a controllable flow generator operable to generate a supply of pressurized breathable gas to be provided to a patient for treatment, a touchless control system associated with at least one control feature adapted to control at least one operating parameter of the flow generator, and a control knob manually movable to enable and disable the touchless control system. The touchless control system includes one or more sensors to detect a patient input and a controller to selectively activate the at least one control feature based on the patient input detected by the one or more sensors.

Yet another aspect of the invention relates to a method for operating a flow generator that generates a supply of pressurized breathable gas to be provided to a patient for treatment. The method includes detecting one or more hand movements of the patient and operating the flow generator at least in part based on the detected hand movements.

Other aspects, features, and advantages of this invention will become apparent from the following detailed description when taken in conjunction with the accompanying drawings, which are a part of this disclosure and which illustrate, by way of example, principles of this invention.

### BRIEF DESCRIPTION OF THE DRAWINGS

The accompanying drawings facilitate an understanding of the various embodiments of this invention. In such drawings:

FIG. 1 is a top perspective view of a flow generator including a touchless control system according to an embodiment of the present invention;

FIG. 2 is a schematic view of a touchless control system according to an embodiment of the present invention;

FIG. 3 is a schematic view of a control knob provided to the flow generator shown in FIG. 1, the control knob configured to enable/disable the touchless control system;

FIG. 4 is a schematic view of a menu tree including a touchless control system with a single swipe model according to an embodiment of the present invention;

FIG. 5 is a schematic view of a menu tree including a touchless control system with a multiple swipe model according to an embodiment of the present invention;

FIG. 6 is a schematic view of breathing apparatus including a retrofit touchless control system according to an embodiment of the present invention; and

FIG. 7 is a schematic view of breathing apparatus including a retrofit touchless control system according to another embodiment of the present invention.

### DETAILED DESCRIPTION OF ILLUSTRATED EMBODIMENTS

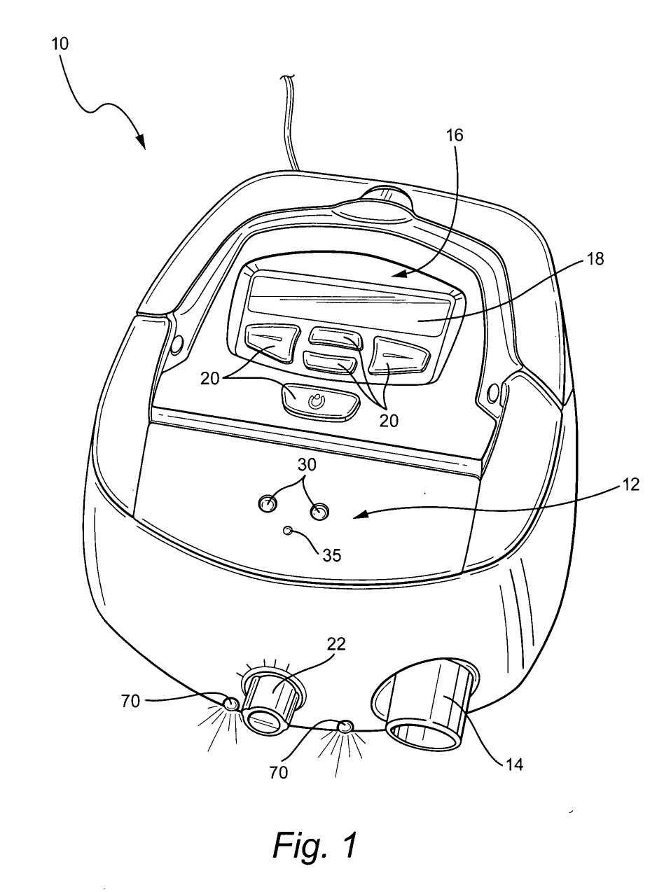

FIG. 1 illustrates a flow generator 10 for a breathing apparatus that includes a touchless control system 12 (also referred to a touchless programmable activator) according to an embodiment of the present invention. The flow generator 10 is structured to generate a supply of pressurized breathable air (e.g., in the range of about 4-20 cmH₂O) to be delivered to a patient for treatment, e.g., of Sleep Disordered Breathing (SDB) with a CPAP or Non-Invasive Positive Pressure Ventilation (NIPPV) device. As discussed in greater detail below, the touchless control system 12 is configured to detect patient input, e.g., patient hand movement, and selectively activate one or more features based on the detected patient input. For example, the touchless control system 12 may adjust (e.g., stop) flow generator operation and/or activate a night light based on the detected patient input. This arrangement facilitates usability of a breathing apparatus to enhance and/or facilitate the treatment session.

### 1. Touchless Control System

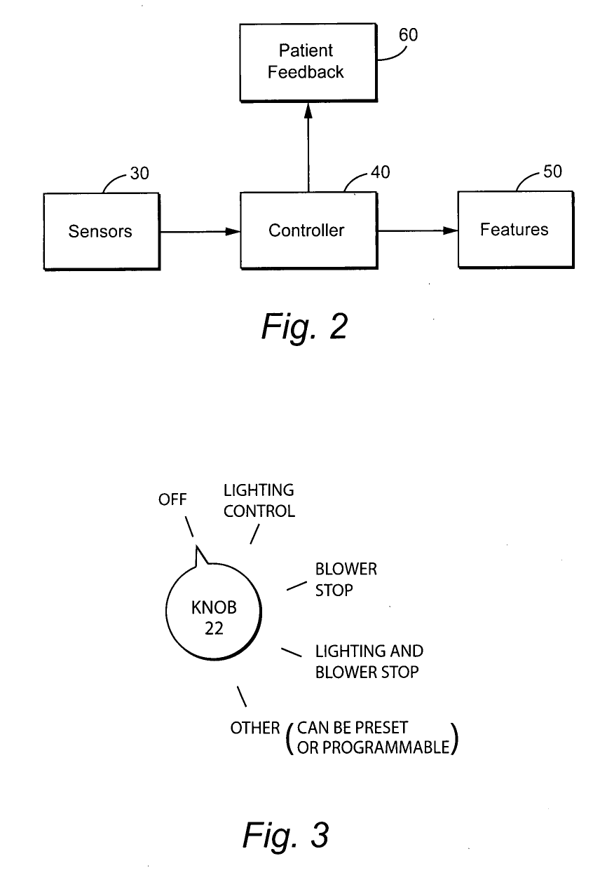

As schematically shown in FIG. 2, the touchless control system 12 includes one or more sensors 30 configured to detect patient input, e.g., patient hand movement such as a hand swiping movement, and a controller 40 to selectively activate one or more features 50 based on the patient input detected by the sensors 30. As described in greater detail below, the controller 40 may provide patient feedback 60, e.g., visual and/or audio feedback, to the patient to acknowledge or confirm that the patient input has been detected.

In the illustrated embodiment, as shown in FIG. 1, the touchless control system 12 includes first and second spaced-apart sensors 30 provided to a top or upper wall of the flow generator 10. This sensor location allows the sensors 30 to easily sense or detect patient input, e.g., hand movement, being performed over the flow generator 10. However, other sensor locations are possible such as those described below.

The sensors 30 generate input, e.g., input signals, representative of the detected patient input, and send the input to the controller 40. The controller 40 is operable to receive input, e.g., input signals, and to selectively activate one or more features 50 based on the input. The input provided by the sensors 30 may be in addition to and/or in lieu of input provided by the control buttons 20. Thus, the sensors 30 allow touchless control or activation of one or more features provided by the flow generator 10.

### 1.1 Touchless Control of Flow Generator

In the illustrated embodiment, the touchless control system 12 is configured to control one or more operating parameters of the flow generator 10. That is, the controller 40 may be configured to adjust flow generator operation based on patient input, e.g., hand movement, detected by the sensors 30.

In its simplest form, the controller 40 may turn off the blower or motor of the flow generator 10. Thus, simply providing a patient input, e.g., hand movement, to the sensors 30 can turn the flow generator 10 on and/or off. In particular, the input may be used to power down the motor of the flow generator.

In alternative embodiments, patient input to the sensors 30 may activate more advanced operating parameters of the flow generator 10. For example, the controller 40 may be configured to control therapy modes, operating pressures, pressure ramp, etc. The touchless control system 12 may mimic a control panel 16 (FIG. 1) and/or a software menu to control the same functions provided by the control panel 16 and/or a software menu.

In general, the touchless control system 12 may be configured to control any operating parameter of the flow generator 10, and these controllable operating parameters may be selected by the patient. That is, the touchless control system 12 may be configurable so that the patient can select the functions he/she wishes to control by the touchless control system 12, e.g., functions frequently used by the patient.

### 1.2 Flow Generator Configuration

As is known in the art, the flow generator 10 is operable to provide a pressurized flow of air or gas at an outlet 14. The supply of pressurized air is delivered to the patient via an air delivery conduit that includes one end communicated to the outlet 14 of the flow generator 10 and an opposite end communicated to a patient interface. The patient interface comfortably engages the patient's face and provides a seal in use. The patient interface may have any suitable configuration as is known in the art, e.g., full-face mask, nasal mask, oro-nasal mask, mouth mask, nasal prongs, etc.

Referring to FIG. 1, the control panel 16 is operable to receive manual input and to control operation of the flow generator 10 based on manual input. In the illustrated embodiment, the control panel 16 includes a display screen 18 and a plurality of control buttons 20, e.g., selection arrow buttons, to selectively activate one or more features provided by the flow generator 10. In addition, the control panel 16 includes a rotatable control knob 22 to enable touchless activation of the flow generator 10.

The touchless control system 12 is incorporated into the flow generator 10 so that the patient can selectively activate one or more features provided by the flow generator without having to select or adjust the control buttons 20. This arrangement reduces the degree of dexterity or coordination previously required to activate such features, thereby encouraging patient compliance.

While the touchless control system 12 is described as being implemented into a flow generator 10 of the type described above, it may be implemented into other flow generator arrangements or other peripheral components (e.g., patient interface) where it is desirable to provide touchless control. That is, the flow generator 10 is merely exemplary, and aspects of the present invention may be incorporated into other suitable arrangements.

### 1.3 Control Knob to Enable/Disable Touchless Control

In the embodiment of FIG. 1, the control knob 22 is provided to enable and disable the touchless control system 12. FIG. 3 illustrates an embodiment of the control knob 22 with several selectable options, including an “off” position (disabling the touchless control system 12) and several “on” positions where the touchless control system 12 is enabled. The “on” positions that are described below are merely exemplary.

For example, when the control knob 22 is in the “off” position, the touchless control system 12 is disabled and the flow generator is controlled using the control buttons 20. When the control knob 22 is rotated to or otherwise in the “lighting control” position, the touchless control system 12 is activated and patient input, e.g., hand movement, may activate a night light, e.g., lights 70 in FIG. 1. Detection of further input, e.g., a hand swipe, will switch off the light. Otherwise, the light can be automatically shut off after a predetermined or selected time period, e.g., 5 minutes, etc.

When the control knob 22 is rotated to or otherwise in the “blower stop” position, the touchless control system 12 is activated and patient input may turn off the flow generator blower (or motor associated therewith) to stop the flow of gas. With the blower off, the patient can the remove the mask and make a trip to the bathroom, kitchen, etc. Upon returning, the patient dons the mask and the “Smartstart™” feature commences therapy. Alternatively, a further hand swipe (or other patient input) can be used to commence therapy.

When the control knob 22 is rotated to or otherwise in the “lighting and blower stop” position, the touchless control system 12 is activated and patient input may turn off the blower/motor and turn on a night light.

When the control knob 22 is rotated to or otherwise in the “other” position, the touchless control system 12 is activated to control more advanced options of the flow generator. The advanced options may be preset or may be programmed by the patient via the control buttons 20.

Thus, the control knob 22 allows selective activation of the touchless control system 12. When enabled, the touchless control system 12 allows touchless activation or touchless access to one or more options provided by the flow generator 10. Touchless activation may be accomplished by patient input such as hand swiping, tapping, and/or voice activation as described below.

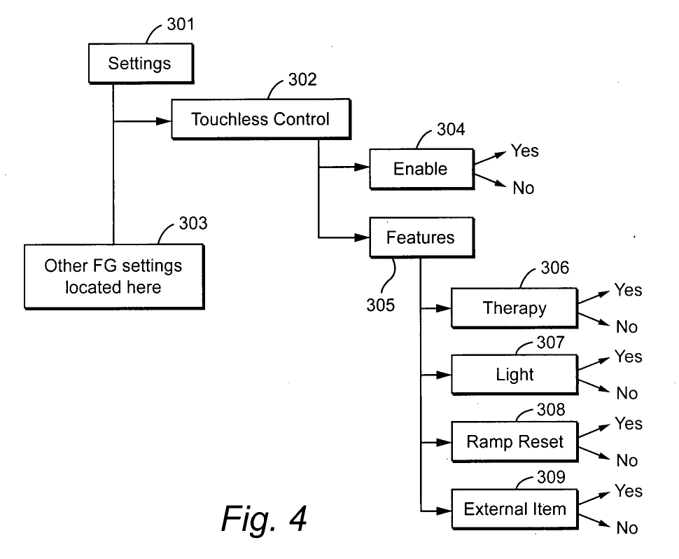

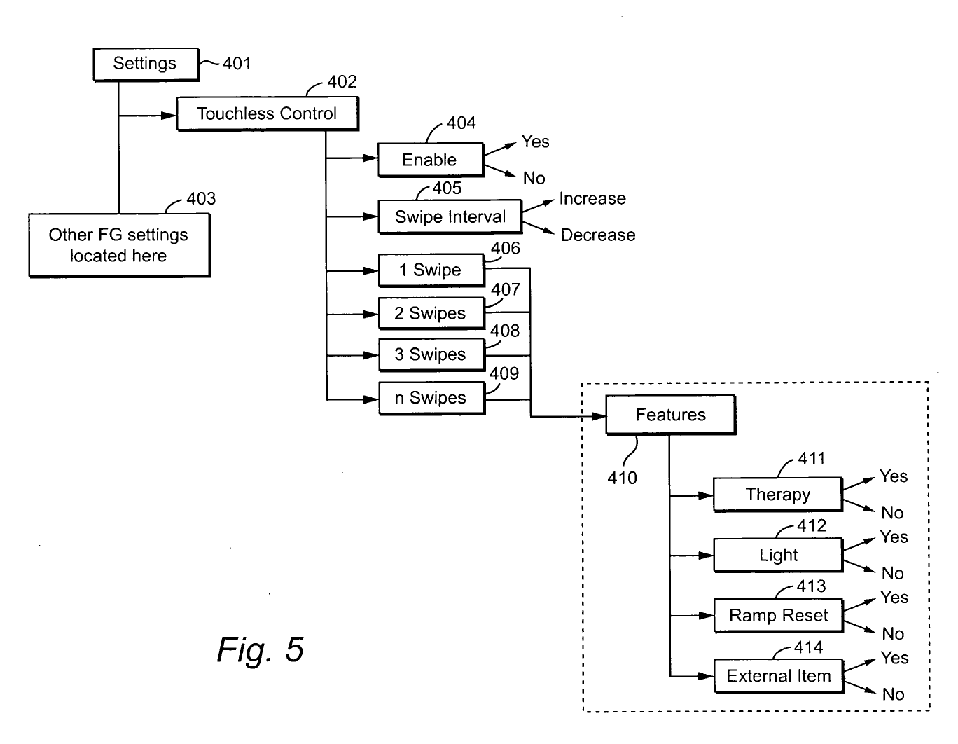

In its simplest form, patient input, e.g., a basic hand swipe, tap, and/or voice command, will activate the option selected by control knob 22. In another embodiment, the control knob 22 may be replaced with software, e.g., touchless control programmed by software via control buttons 20, discussed in further detail in relation to FIGS. 4 and 5 for example. In yet another embodiment, the control knob 22 and software may both be used to establish touchless control. For example, the control knob 22 may be moved to the “other” option to activate touchless control, and the advanced options are programmed or selected by software via control buttons 20.

### 1.4 Touchless Control of Other Features

The touchless control system 12 may be configured to activate other features besides those associated with the flow generator blower.

### 1.4.1 Night Light

As shown in FIG. 1, the flow generator 10 includes a “night light” or “foot light” for illuminating the ground adjacent the flow generator 10. Specifically, first and second spaced-apart light sources 70, e.g., light bulbs or LEDs, are provided to a side wall of the flow generator 10 adjacent the ground. When activated, the light sources 70 act as a “night light” or “foot light” to illuminate the ground and/or surface adjacent the flow generator 10 and facilitate a patient's movement in the dark.

In an embodiment, the light sources 70 may be activated by the touchless control system 12. That is, patient input, e.g., hand movement over the sensors 30, may signal the controller 40 to turn the light sources 70 on and/or off.

### 1.4.2 Remote Lighting

The touchless control system 12 may be configured to activate other light sources remote from the flow generator 10. For example, the touchless control system 12 may be configured to illuminate a light source provided to the patient interface as described in PCT Application No. PCT/AU2005/000704, entitled “Position Sensitive Illumination”, the entirety incorporated herein by reference. In another embodiment, the touchless control system 12 may be configured to activate a hall light, bathroom light, bedside lamp, etc.

### 1.4.3 Rheostat or Dimmer

In each embodiment, the touchless control system 12 may be configured to control the magnitude/intensity of the illuminated light, e.g., a rheostat or dimmer. For example, the patient may move his/her hand toward and away from the sensors 30 to increase and decrease the lighting magnitude, i.e., hand distance from sensor controls the magnitude of the given function, as described in greater detail below.

### 1.4.4 Additional Power Sockets

In another embodiment, the flow generator 10 may include additional power sockets to provide power to other devices, e.g., lamp, fan, television. The touchless control system 12 may be configured to control power being supplied to these other devices. Thus, patient input to the sensors 30 may signal the controller 40 to turn the other device connected to the flow generator on and/or off.

### 1.5 Hand Movement

The one or more sensors 30 may be configured to sense various hand movement methods or arrangements so that specific hand movement methods or arrangements can be associated with specific features provided to the flow generator 10.

### 1.5.1 Single Swipe Method

In an embodiment, a single swipe or hand motion over the sensors 30, e.g., moving hand across the sensors 30, may signal to controller 40 to activate the programmed feature.

FIG. 4 is a menu tree for a flow generator including a touchless control system with a single swipe model. As illustrated, the control panel of the flow generator may include a settings option 301 that controls the settings for the touchless control 302 and other flow generator parameters 303. Under to touchless control option 302, the patient may select to enable or disable touchless control at 304. If touchless control is enabled at 304, then the patient enters a features option 305 to select which one of the features provided by the flow generator to activate by touchless control. In the illustrated embodiment, the patient has four feature options, e.g., therapy 306, light 307, ramp reset 308, and external item 309, such as a radio, TV, etc. However, more or less feature options may be available. In use, simply swiping or waving a hand over the sensors 30 signals the controller 40 to activate the selected feature option, e.g., start/stop therapy 306, turn light on/off 307, reset ramp 308, activate external item 309.

The menu tree of FIG. 4 is the software analog to the control knob 22. In both systems, the patient decides whether to enable touchless control and identifies the feature or features to be activated by touchless control.

### 1.5.2 Multiple Swipe Method

In another embodiment, different swipe numbers, e.g., one or more subsequent hand swipes across the sensors 30, may correspond to different features. Specifically, each programmed feature may be controlled by a specific number of swipes. For example, one swipe would activate the first function, two swipes would activate the second function, three swipes would activate the third function, etc. Audio and/or visual patient feedback may be provided after each swipe.

FIG. 5 is a menu tree for a flow generator including a touchless control system with a multiple swipe model. As illustrated, the control panel of the flow generator may include a settings option 401 that controls the settings for the touchless control 402 and other flow generator parameters 403. Under to touchless control option 402, the patient may select to enable or disable touchless control at 404. If touchless control is enabled at 404, then the patient enters a swipe interval option 405 to select the swipe interval, e.g., increase or decrease interval between successive swipes. After the swipe interval is selected, then the patient selects which features under the features option 410 to coordinate with a specific swipe number at 406, 407, 408, 409 that will activate the selected feature. The swipe numbers may be selected by the patient and may include 1 swipe at 406, 2 swipes at 407, 3 swipes at 408, and “n” swipes at 409. Each swipe number will have the same options, i.e., any of the feature options may be programmed or associated with any suitable swipe number. Thus, 1 to “n” swipes may be programmed with a respective one of any of the features.

In the illustrated embodiment, the patient has four feature options, e.g., therapy 411, light 412, ramp reset 413, and external item 414, to associate with a respective one of 1 to “n” swipes. However, more or less options may be available. In use, swiping a hand once over the sensors 30 signals the controller 40 to activate the feature associated with one swipe, swiping a hand twice over the sensors 30 signals the controller 40 to activate the feature associated with two swipes, waving a hand “n” times over the sensors 30 signals the controller 40 to activate the feature associated with “n” swipes, etc.

### 1.5.3 Morse Code Style Swiping

In yet another embodiment, different swipe numbers along with different swipe speeds may correspond to different features. Specifically, each programmed feature may be controlled by a specific number of swipes in combination with the speed of each swipe. Thus, the sensors are configured to detect a slow swipe from a quick swipe as well as monitor successive swipes. This is similar to Morse code in which strings of short and long signals (e.g., strings of dots and dashes) represent letters and numbers.

In this method, there are two ways to send a command to the sensors, i.e., a normal or quick swipe of the hand and a slow swipe of the hand. The slow swipe may be as slow as holding one's hand over the sensors for a couple seconds. However, the speed of the slow swipe may be settable.

Having two ways to send a command to the sensors greatly enhances the efficiency of control. For example, in the multiple swipe method, one would need to swipe 6 times to activate the 6^{th} feature. With Morse code style swiping or waving, the command string can be reduced. For example, the following illustrates a command list for controlling 6 features or functions. As illustrated, only 2 swipes are needed to activate the 6^{th} function rather than 6 swipes with the multiple swipe method. However, other combinations are possible.

Function 1: a normal wave

Function 2: a slow wave

Function 3: 2 short waves

Function 4: 1 short wave followed by 1 slow wave

Function 5: 1 slow wave followed by 1 short wave

Function 6: 2 slow waves

### 1.5.4 Vertical and Horizontal Wave Detection

In yet another embodiment, the commands may be distance or directional dependent. Specifically, the sensors 30 may be configured to detect hand motion in the vertical direction, e.g., towards and away from the sensors, and detect hand motion in the horizontal direction, e.g., across the sensors. Thus, the sensors detect the location of one's hand, and signal functional adjustment based on the direction of the wave or swipe.

In its simplest form, the sensors 30 may be capable of detecting distance to an object (e.g., such as ultrasonics) so as to detect when ones hand is moving up or moving down above the sensors 30 (e.g., vertical detection). In an embodiment, moving up or away from the sensors may be associated with an “up” command or arrow to adjust a programmed feature up, e.g., raise ramp time. Similarly, moving down or towards the sensors may be associated with a “down” command or arrow to adjust a programmed feature down, e.g., lower ramp time.

With the addition of left and right detection (e.g., horizontal detection), it is possible to further enhance the capacity of touchless control. In an embodiment, moving right of the sensors may be associated with a “right arrow” command to adjust or select a programmed feature to the right. Similarly, moving left of the sensors may be associated with a “left arrow” command to adjust or select a programmed feature to the left.

In the control panel 16 described above, the four control buttons 20 adjacent the screen 18 may represent up, down, left, and right arrows or commands that are used to modify settings, browse through menus, etc. Each of these four commands may be associated with a directional hand movement as described above to provide touchless control of the flow generator, e.g., upwards movement for up arrow, downwards movement for down arrow, left movement for left arrow, and right movement for right arrow.

In another embodiment, hand distance from the sensors may control magnitude and/or speed of a given function. For example, the patient may move his/her hand toward and away from the sensors to increase and decrease lighting magnitude, blower speed, etc.

### 1.6 Patient Feedback

Patient feedback to confirm detection of patient input may be visual and/or audio feedback.

### 1.6.1 Visual Feedback

In the illustrated embodiment, the flow generator 10 includes a light source 35 that provides patient feedback to the patient to acknowledge or confirm that patient input, e.g., hand movement, has been detected by the sensors 30. Specifically, the light source 35, e.g., light bulb or LED, is provided to a top or upper wall of the flow generator 10 adjacent the sensors 30. This light source location allows the patient to easily view illumination of the light source 35. However, other light source locations are possible.

When activated, the light source 35 acts as a confirmation signal to confirm detection of patient input by the sensors 30, e.g., confirm registration of a swipe. For example, a green light may confirm detection and a red light may indicate no detection. The light source may illuminate for a predetermined period of time and/or flash on and off.

### 1.6.2 Audio Feedback

In another embodiment, patient feedback may be provided by an audible signal, e.g., voice feedback or one or more beeps. The audible signal may be in lieu of or in addition to the light source. For example, a computer generated voice may confirm the selection by saying “power off” or “ramp down”.

### 1.6.3 Audio Confirmation or Activation

In still another embodiment, the patient feedback may require audio feedback from the patient, e.g., a simple yes or no. For example, the patient may perform a hand movement to control flow generator operation. The flow generator confirms the hand movement with an audible response, e.g., “you have selected power off”. If this command is correct, then the patient simply says “yes”. Following the patient's audio confirmation, the flow generator will proceed to power off or otherwise perform the desired command. Thus, the flow generator may operate based on both patient hand movements and patient audible commands. In an embodiment, the flow generator may be primarily voice activated, e.g., operating parameters controlled by voice commands.

### 1.6.4 Projected Display Screen

In yet another embodiment, information on the display screen 18 may be projected onto the ceiling or other adjacent surface. For example, see projection unit disclosed in U.S. Provisional Application No. 60/703,432, entitled “Lifestyle Flow Generator and Mask System,” the entirety incorporated herein by reference. Thus, the patient can view information on the projected display screen 18 to confirm a given command.

### 1.6.5 Time Delay

In an embodiment, a time delay may be provided between patient feedback and actual activation of the selected feature. That is, the touchless control system may acknowledge a command then wait a predetermined period of time, e.g., 1 minute, 10 minutes, etc., before executing the command. The predetermined period of time may be programmed by the patient and/or preset based on the command.

### 1.7 Sensor Location and Implementation

In the illustrated embodiment, the sensors 30 are provided to a top or upper wall of the flow generator 10. However, other sensor locations are possible to provide touchless control.

For example, hand motion sensors may be provided to the patient interface, e.g., on the mask or headgear, and input may be transmitted, e.g., by wire or wirelessly, to the controller to control flow generator operation.

The touchless control system 12 may be implemented using hardware, e.g., control knob 22, or software, e.g., added to the menu structure of the control panel, as described above.

### 1.8 Retrofit

In the illustrated embodiment, the touchless control system is implemented into the flow generator 10, i.e., during original manufacture. However, the touchless control system may be retrofit, e.g., retrofit to an existing flow generator. That is, the touchless control system may be in the form of a separate unit that can be retrofit to an existing breathing arrangement.

### 1.8.1 Touchless Controller to Control Air Flow

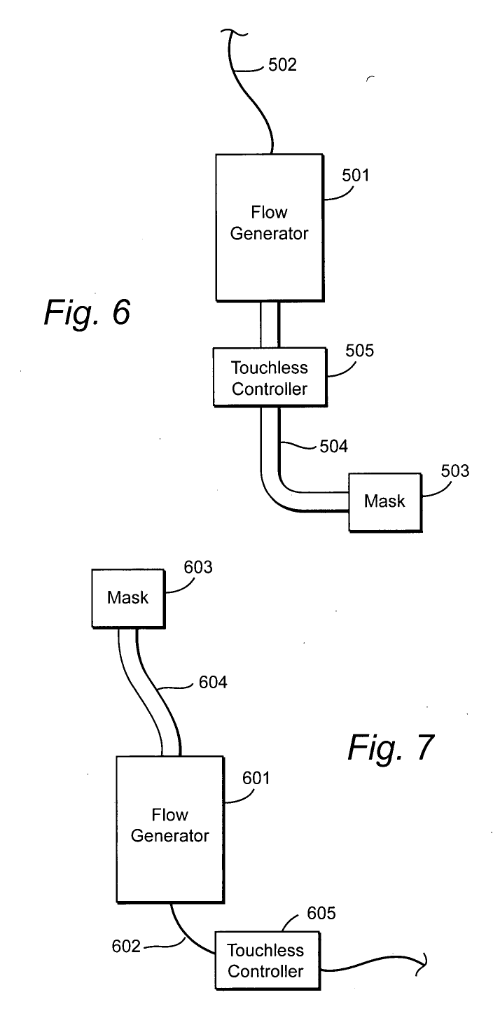

For example, FIG. 6 illustrates a breathing apparatus including a flow generator 501 with power cord 502, a mask 503, and tubing 504 to communicate the flow generator 501 and the mask 503. As illustrated, a touchless controller 505 is attachable along the tubing 504. The touchless controller 505 includes sensors for detecting input, e.g., hand motion, and a valve to control air flow. In use, input detected by the sensors will signal a controller to activate the valve which will permit or stop air flow along the tubing 504. Thus, the touchless controller 505 controls air flow, thereby basically controlling whether the flow generator 10 is on and/or off.

### 1.8.2 Touchless Controller to Power Down Blower

FIG. 7 illustrates another embodiment of a breathing apparatus including a flow generator 601 with power cord 602, a mask 603, and tubing 604 to communicate the flow generator 601 and the mask 603. As illustrated, a touchless controller 605 is attachable along the power cord 602. The touchless controller 605 includes sensors for detecting input, e.g., hand motion, and a controller to turn off the blower or motor associated therewith. In use, input detected by the sensors will signal the controller to power down the blower of the flow generator 601. Thus, the touchless controller 605 controls blower operation to control whether the flow generator 10 provides air flow.

### 1.9 Sensor Configuration

The sensors 30 of the touchless control system 12 may have any suitable configuration for sensing input such as hand motion. However, some sensor configurations may be more suitable for detecting certain hand movement arrangements, e.g., Morse code style, vertical and horizontal wave detection, etc.

Exemplary sensor configurations include: change of state sensors, optical sensors, US (ultrasonic) sensors, IR (infra-red) sensors, body heat (passive IR) sensors, and microwave detectors (body heat and movement). The microwave detectors may be embedded within the flow generator and do not need a direct line of view, e.g., like US sensors.

Also, the sensors may be voice sensors for voice activation. In addition, the sensors may be configured to sense tapping or vibrations for tapping activation.

### 1.10 Condensation Control

The touchless control system 12 may be used in conjunction with condensation control, as discussed in relation to U.S. patent application Ser. No. 11/207,007, filed Aug. 19, 2005, incorporated herein by reference in its entirety. In an embodiment, condensation control may use the condensation sensor for the touchless control system. Condensation control monitors condensation in the patient interface, e.g., mask, and adjusts humidity accordingly. For example, condensation control may use a reflective arrangement wherein humidity adjustment is based on a signal directed towards the mask. If the signal is reflected, then condensation is high and the humidity is lowered. Likewise, if the signal passes through the mask, then condensation is suitable. In an embodiment, humidity adjustment may be completed using touchless control.

### 1.11 Alternative Embodiments of Touchless Control

In the illustrated embodiment, the touchless control system uses sensors that detect input such as hand motion. However, other sensors may be used to signal a controller to activate a feature.

In an embodiment, the sensor may be a pressure-activated sensor that is configured to detect pressure applied by the patient. For example, the pressure-activated sensor may be embedded with the patient's pillow to detect whether or not the patient's head is on the pillow. When the patient's head is not on the pillow (e.g., indicating that the patient is up from the bed), the sensor may signal the controller to turn off the flow generator and activate a night light for example.

In another embodiment, the pressure-activated sensor may be provided in a floor pad to be positioned adjacent the patient's bed. When the patient stands on the floor pad (e.g., indicating that the patient is up from the bed), the sensor may signal the controller to turn off the flow generator and activate a night light for example.

In yet another embodiment, the sensor may be configured to detect tapping or knocking, e.g., on the flow generator, mattress, or bedside table. Similar to the multiple swipe method, each programmed feature may be controlled by a specific number of taps. For example, one tap would activate the first function, two taps would activate the second function, three taps would activate the third function, etc.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments. In addition, while the invention has particular application to patients who suffer from OSA, it is to be appreciated that patients who suffer from other illnesses (e.g., congestive heart failure, diabetes, morbid obesity, stroke, barriatric surgery, etc.) can derive benefit from the above teachings. Moreover, the above teachings have applicability with patients and non-patients alike in non-medical applications.

What is claimed is:

1. An air delivery system, comprising:
a controllable flow generator operable to generate a supply of pressurized breathable gas to be provided to a patient for treatment; and
a touchless control system associated with at least one control feature adapted to control at least one operating parameter of the flow generator, the touchless control system including one or more sensors to detect patient hand movement and a controller to selectively activate the at least one control feature based on the patient hand movement detected by the one or more sensors.

2. The air delivery system according to claim 1, wherein the touchless control system provides patient feedback to confirm detection of a patient hand movement.

3. The air delivery system according to claim 2, wherein the patient feedback includes at least one of a light source and an audible signal.

4. The air delivery system according to claim 1, wherein the one or more sensors are provided to a top wall of the flow generator.

5. The air delivery system according to claim 1, wherein the at least one control feature includes power to the flow generator blower, therapy mode, operating pressure, and pressure ramp.

6. The air delivery system according to claim 1, wherein the at least one control feature is selectable by the patient.

7. The air delivery system according to claim 1, wherein the patient hand movement includes one or more hand swipes or waves over the sensors.

8. The air delivery system according to claim 7, wherein the patient hand movement includes a single hand swipe, the sensors being configured to detect the single hand swipe and signal the controller to selectively activate the at least one control feature associated with the single hand swipe.

9. The air delivery system according to claim 7, wherein the patient hand movement includes multiple hand swipes, and the at least one control feature is associated with a specific number of successive hand swipes.

10. The air delivery system according to claim 9, wherein the sensors are configured to detect specific numbers of successive hand swipes and signal the controller to selectively activate the at least one control feature associated with the specific number detected.

11. The air delivery system according to claim 9, wherein the sensors are configured to distinguish between slow and quick swipes.

12. The air delivery system according to claim 1, wherein the patient hand movement includes hand movement in vertical and/or horizontal directions with respect to the sensors.

13. The air delivery system according to claim 12, wherein hand movement in vertical directions acts as a rheostat or dimmer to control the magnitude of a light associated with the touchless control system.

14. The air delivery system according to claim 1, wherein the touchless control system is provided as a separate unit that is retrofit to the flow generator.

15. The air delivery system according to claim 14, wherein the separate unit is attachable along air delivery tubing, the separate unit including a valve to control air flow based on the detected patient hand movement.

16. The air delivery system according to claim 14, wherein the separate unit is attachable along a power supply cord, the separate unit configured to control power supply to the blower based on the detected patient hand movement.

17. The air delivery system according to claim 1, wherein the sensors are in the form of change of state sensors, optical sensors, ultrasonic sensors, infra-red sensors, body heat sensors, and/or microwave detectors.

18. The air delivery system according to claim 1, further comprising a control knob manually movable to enable and disable the touchless control system.

19. The air delivery system according to claim 18, wherein the control knob is selectively movable to one or more enabled positions, each said enabled position corresponding to the control feature or a plurality of control features associated with the touchless control system.

20. An air delivery system, comprising:
a controllable flow generator operable to generate a supply of pressurized breathable gas to be provided to a patient for treatment;
a touchless control system associated with at least one control feature adapted to control at least one operating parameter of the flow generator, the touchless control system including one or more sensors to detect a patient input and a controller to selectively activate the at least one control feature based on the patient input detected by the one or more sensors; and
a control knob manually movable to enable and disable the touchless control system.

21. The air delivery system according to claim 20, wherein the control knob is selectively movable to one or more enabled positions, each said enabled position corresponding to the control feature or a plurality of control features associated with the touchless control system.

22. The air delivery system according to claim 20, wherein the patient input includes hand swiping, tapping, and/or voice command.

23. The air delivery system according to claim 20, wherein the at least one control feature includes power to the flow generator blower, therapy mode, operating pressure, and pressure ramp.

24. A breathing apparatus comprising:
an air delivery system according to claim 1;
a patient interface engagable with a patient's face to provide a seal;
an air delivery conduit provided between the air delivery system and the patient interface to deliver the supply of pressurized air from the air delivery system to the patient interface.

25. A method for operating a flow generator that generates a supply of pressurized breathable gas to be provided to a patient for treatment, the method comprising:
detecting one or more hand movements of the patient; and
operating the flow generator at least in part based on the detected hand movements.

26. The method according to claim 25, further comprising swiping a hand over a sensor and turning the flow generator or motor associated therewith off.
